(19) United States
(12) Patent Application Publication
(10) Pub. No.: US 2024/0384212 A1
(43) Pub. Date: Nov. 21, 2024

(54) CELL CULTURE MEMBRANE AND CELL CULTURE METHOD

(71) Applicant: TOYODA GOSEI CO., LTD., Kiyosu-shi (JP)

(72) Inventors: Seitaro TAKI, Kiyosu-shi (JP); Yasuhiko SHINODA, Kiyosu-shi (JP); Kosuke IWATA, Kiyosu-shi (JP); Sayaka HINO, Nagoya-shi (JP)

(21) Appl. No.: 18/628,143

(22) Filed: Apr. 5, 2024

(30) Foreign Application Priority Data
May 19, 2023 (JP) 2023-082736

(51) Int. Cl. C12M 1/12; C08L 75/04; C12M 1/00

(52) U.S. Cl. CPC C12M 25/02; C08L 75/04; C12M 23/20

(57) ABSTRACT

A cell culture membrane includes a resin porous membrane formed of a resin and having a plurality of pores that is open on at least one surface, in which at least some of the pores are through-holes that penetrate in a membrane thickness direction, and a solid coating material that is applied to any one surface of the resin porous membrane and suppresses cells from passing through the through-holes.

### TECHNICAL FIELD

The present invention relates to a cell culture membrane and a cell culture method.

### BACKGROUND ART

Cell culture is performed in various fields such as drug discovery and regenerative medicine. Usually, cells are generally cultured using a cell culture membrane as a scaffold and by adding a medium containing nutrients thereonto.

The present applicants have developed and disclosed, as a previous novel cell culture membrane, a porous membrane formed of a thermosetting resin and having a plurality of pores that open on at least one surface, in which at least some of the pores are through-holes that penetrate in a membrane thickness direction (Patent Literature 1). In this example, polyurethane is used as the thermosetting resin. In addition, the pores and through-holes have cone shapes, and the average pore size of the through-holes on the opening side is 5 μm to 15 μm, but becomes smaller toward the other side of the penetrating through-holes (for example, around 3 μm). Therefore, it is difficult to pass cells through the through-holes.

According to this polyurethane porous membrane, it is possible to culture different types of cells A and B on both surfaces thereof and analyze interactions between the different types of cells. Specifically, the cells A are seeded on one surface to block pores, and the other cells B are seeded on the other surface to perform two-layer culture.

However, when culturing of cells (the cells A alone or the cells B alone) in a monolayer only on one surface of the polyurethane porous membrane is desired, the cells may go around to the opposite surface through the through-holes. This is because some cells have an ability to migrate or invade, and the cells contract and pass through the through-holes. If the number of through-holes is larger and the diameter is larger, the number of cells that pass through also increases. Therefore, with this polyurethane porous membrane, it is difficult to perform monolayer culture as opposed to two-layer culture.

Here, Patent Literature 2 discloses a two-layer flow path device in which a lower layer microchannel chip and an upper layer microchannel chip are stacked across a porous membrane having pores with a size that does not allow cells to pass through, and it is said that, when cells are seeded by coating an upper layer flow path with fibronectin and introducing a cell suspension, no cells are observed in the lower layer. Examples of materials of the porous membrane include polyethylene terephthalate, nylon, nitrocelluloses, polydimethylsiloxane, and collagen Vitrigel. The fibronectin coating is described as mainly promoting cell adhesion, but there is no description that it suppresses cells from passing through the through-holes, and it is not clear whether the fibronectin is a liquid or a solid.

### CITATION LIST

### Patent Literature

[Patent Literature 1] Japanese Unexamined Patent Application Publication No. 2017-29092 (JP 2017-29092 A)

[Patent Literature 2] Japanese Unexamined Patent Application Publication No. 2020-188723 (JP 2020-188723 A)

### SUMMARY OF THE INVENTION

### Technical Problem

Here, an object of the present invention is to, when cells are cultured in a monolayer only on one surface of a polyurethane porous membrane having through-holes, suppress the cells from going around to the other side through the through-holes, and to make it possible to perform, for example, monolayer culture as opposed to two-layer culture.

### Solution to Problem

[1] A cell culture membrane including a resin porous membrane formed of a resin and having a plurality of pores that is open on at least one surface, in which at least some of the pores are through-holes that penetrate in a membrane thickness direction, and a coating material that is applied to any one surface of the resin porous membrane and suppresses cells from passing through the through-holes.

### Effects

The coating material appropriately fills and blocks the through-holes of the resin porous membrane, and thus cells seeded on one surface of the resin porous membrane are suppressed from passing through the through-holes and going around to the opposite surface.
If the coating material completely blocks the through-holes, neither cell nuclei nor pseudopods go around.
If the coating material blocks the through-holes to some extent, cell nuclei do not go around, but pseudopods in the cytoplasmic part may go around (feed around).

[2] The cell culture membrane according to [1],
wherein the resin is a polyurethane, and the pores and the through-holes have a cone shape.
Polyurethanes have excellent elasticity and strength and are flexible, and are not only suitable for cell culture, but also allow easy formation of cone-shaped through-holes by a method to be described below.

[3] The cell culture membrane according to [1] or [2],
wherein the coating material is a dried glycoprotein product.
Since it has good cell adhesion and adhesion to the membrane, it is difficult to peel off.
If the coating material is a dried product (semi-dry state), water is removed and adhesive strength with respect to the resin porous membrane increases (blocks the through-holes).

[4] The cell culture membrane according to [1] or [2],
wherein the coating material is a protein gel.
Since it has good cell adhesion and adhesion to the membrane, it is difficult to peel off.
When the coating material is made into a gel and made sticky, the adhesive strength with respect to the membrane increases.
Since the gel has a small amount of syneresis, syneresis does not weaken adhesiveness between the membrane and the gel.

[5] The cell culture membrane according to [1] or [2],
wherein the coating material is a polysaccharide gel.
Since it has good cell adhesion and adhesion to the membrane, it is difficult to peel off.

[6] A cell culture method using a resin porous membrane formed of a resin and having a plurality of pores that is open on at least one surface, in which at least some of the pores are through-holes that penetrate in a membrane thickness direction, the cell culture method including
coating any one surface of the resin porous membrane with a coating material that suppresses cells from passing through the through-holes to form a cell culture membrane, and performing monolayer culture of cells on one surface of the cell culture membrane.

[7] A cell culture method using first and second resin porous membranes formed of a resin and having a plurality of pores that is open on at least one surface, in which at least some of the pores are through-holes that penetrate in a membrane thickness direction, the cell culture method including:
a step of using the first resin porous membrane as a cell culture membrane and performing two-layer culture of different types of cells on both surfaces of the cell culture membrane; and
a step of coating any one surface of the second resin porous membrane with a coating material that suppresses cells from passing through the through-holes to form a cell culture membrane, and performing monolayer culture of cells on one surface of the cell culture membrane.

### Advantageous Effects of Invention

According to the present invention, when cells are cultured in a monolayer only on one surface of a polyurethane porous membrane having through-holes, it is possible to suppress the cells from going around to the other side through the through-holes, and for example, to perform monolayer culture as opposed to two-layer culture.

### BRIEF DESCRIPTION OF DRAWINGS

FIGS. 1A to 1C show a PU porous membrane in an example, FIG. 1A is a cross-sectional view, FIG. 1B is a cross-sectional view when the PU porous membrane is fixed to a bottom of an insert, and FIG. 1C is a perspective view showing an insert and a well;

FIGS. 2A to 2C show a cell culture membrane of Sample 2 using the same membrane, FIG. 2A is a cross-sectional view when a coating material solution is applied, FIG. 2B is a cross-sectional view when a coating material becomes a dried product, and FIG. 2C is a cross-sectional view during cell culture;

FIGS. 3A and 3B show a cell culture membrane of Sample 6 coated with the same membrane, FIG. 3A is a cross-sectional view when a coating material solution is solidified into a thick gel in the well, and FIG. 3B is a cross-sectional view during cell culture;

FIGS. 4A and 4B show a cell culture membrane of Sample 7 coated with the same membrane, FIG. 4A is a cross-sectional view when a coating material solution is added, and FIG. 4B is a cross-sectional view during cell culture;

FIG. 5A is a DAPI-stained image and FIG. 5B is a phalloidin-stained image of Sample 1 using the same membrane;

FIG. 6A is a DAPI-stained image and FIG. 6B is a phalloidin-stained image of Sample 2 coated with the same membrane;

FIG. 7A is a DAPI-stained image and FIG. 7B is a phalloidin-stained image of Sample 3 coated with the same membrane;

FIG. 8A is a DAPI-stained image and FIG. 8B is a phalloidin-stained image of Sample 4 coated with the same membrane;

FIG. 9A is a DAPI-stained image and FIG. 9B is a phalloidin-stained image of Sample 5 coated with the same membrane;

FIG. 10A is a DAPI-stained image and FIG. 10B is a phalloidin-stained image of one sample of Sample 6 coated with the same membrane;

FIG. 11A is a DAPI-stained image and FIG. 11B is a phalloidin-stained image of another sample of Sample 6 coated with the same membrane;

FIG. 12A is a DAPI-stained image and FIG. 12B is a phalloidin-stained image of Sample 7 coated with the same membrane;

FIG. 13A is a DAPI-stained image and FIG. 13B is a phalloidin-stained image of Sample 8 coated with the same membrane;

FIG. 14 is a graph showing the variation in the TEER value of Sample 1 using the PU porous membrane/PC porous membrane; FIG. 15 is a graph showing the variation in the TEER value of Sample 2 coated with the PU porous membrane/PC porous membrane;

FIG. 16 is a graph showing the variation in the TEER value of Sample 3 coated with the PU porous membrane/PC porous membrane;

FIG. 17 is a graph showing the variation in the TEER value of Sample 4 coated with the PU porous membrane/PC porous membrane;

FIG. 18 is a graph showing the variation in the TEER value of Sample 5 coated with the PU porous membrane/PC porous membrane;

FIG. 19 is a graph showing the variation in the TEER value of Sample 6 coated with the PU porous membrane/PC porous membrane;

FIG. 20 is a graph showing the variation in the TEER value of Sample 7 coated with the PU porous membrane/PC porous membrane;

FIG. 21 is a graph showing the variation in the TEER value of Sample 8 coated with the PU porous membrane/PC porous membrane; and

FIG. 22 is a graph showing the TEER value that varies between two samples of the same Sample 6.

### DESCRIPTION OF EMBODIMENTS

### 1. Resin Porous Membrane

The resin material for the porous membrane is not particularly limited, and in addition to polyurethane (PU), polycarbonate (PC), polyethylene terephthalate (PET), and polyimide (PI), which are general track-etched membrane materials used in the cell culture container, may be exemplified. Polyurethanes have excellent elasticity and strength and are flexible, and are not only suitable for cell culture, but also allow easy formation of cone-shaped through-holes by a method to be described below.

The thickness of the resin porous membrane is not particularly limited, and is preferably 1 μm to 20 μm and more preferably 5 μm to 10 μm.

### 2. Pores and Through-Holes

The number of pores is not particularly limited, and is preferably 1E+04 to 1E+06/cm².
The average pore size of pore openings is not particularly limited, and is preferably 0.1 μm to 100 μm and more preferably 1 μm to 30 μm.
The average pore size on the other side of the through-holes is not particularly limited, and is preferably 0.1 μm to 30 μm and more preferably 1 μm to 10 μm.

### 3. Coating Material

The coating material is not particularly limited as long as it suppresses cells from passing through the through-holes, and examples thereof include dried glycoprotein products, protein gels, and polysaccharide gels.
Glycoproteins are not particularly limited, and examples thereof include fibronectin, adipine, mucin, proteoglycan, entactin, and vitronectin.
Proteins are not particularly limited, and examples thereof include collagen and gelatin.
Polysaccharides are not particularly limited, and examples thereof include agarose, pectin, carboxymethyl cellulose, xanthan gum, gellan gum, and carrageenan.

### EXAMPLE

Next, examples of the present invention will be described with reference to the drawings. Here, structures, materials, shapes and sizes of respective parts of the examples are only examples, and can be appropriately changed without departing from the spirit and scope of the invention.

### <1>Preparation of Cell Culture Membrane Sample Using PU Porous Membrane

A polyurethane (PU) porous membrane was prepared by a method of an example described in Patent Literature 1.
Specifically, a polyether polyol (a polypropylene ethylene polyol (PPG) with a number-average molecular weight of about 4,000 and a hydroxyl value of 37) was used as a polyol, a polyol modified product of diphenylmethane diisocyanate (MDI) containing 28.0 mass % of isocyanate groups (NCO) at the molecular end was used as an isocyanate, diethylene glycol (DEG) was used as a crosslinking agent, tetrahydrofuran (THF) was used as a diluting solvent, and ultrapure water (Milli-Q water) was used as a modifier.

An uncured PU raw material layer formed of these raw materials was formed on a polypropylene (PP) film, which was a film-forming substrate, by a spin coating method. Then, the PU raw material layer was cured while supplying water vapor to the uncured PU raw material layer to prepare a PU porous membrane.

Water vapor was supplied to the uncured PU raw material layer as follows. That is, water was put into a closed container, which was placed in a thermostatic chamber set at a curing temperature (for example, 60° C.), and heating was performed until the temperature of water in the closed container reached the curing temperature. Then, the uncured PU layer was fixed to the back side of the lid of the closed container, along with the film-forming substrate, and the uncured PU layer was placed so that it faced water in the closed container. Then, the closed container was returned to the thermostatic chamber, and the uncured PU raw material layer was subjected to a curing reaction while being exposed to saturated water vapor at the curing temperature.

In this manner, by adjusting conditions selected from the reaction temperature, the reaction time, and the PU raw material composition in the curing reaction when the PU porous membrane was prepared, it was possible to control the shape of the PU porous membrane. Specifically, by changing the conditions, the diameter of pores formed in the PU porous membrane, the depth of the pores (whether at least some of the pores were through-holes), the shapes of the pores and the like could be changed. Here, PU porous membranes were prepared by variously changing the conditions, the membrane thickness and average pore diameter of the obtained PU porous membrane were measured, and the shapes of the pores and the presence of through-holes were checked.

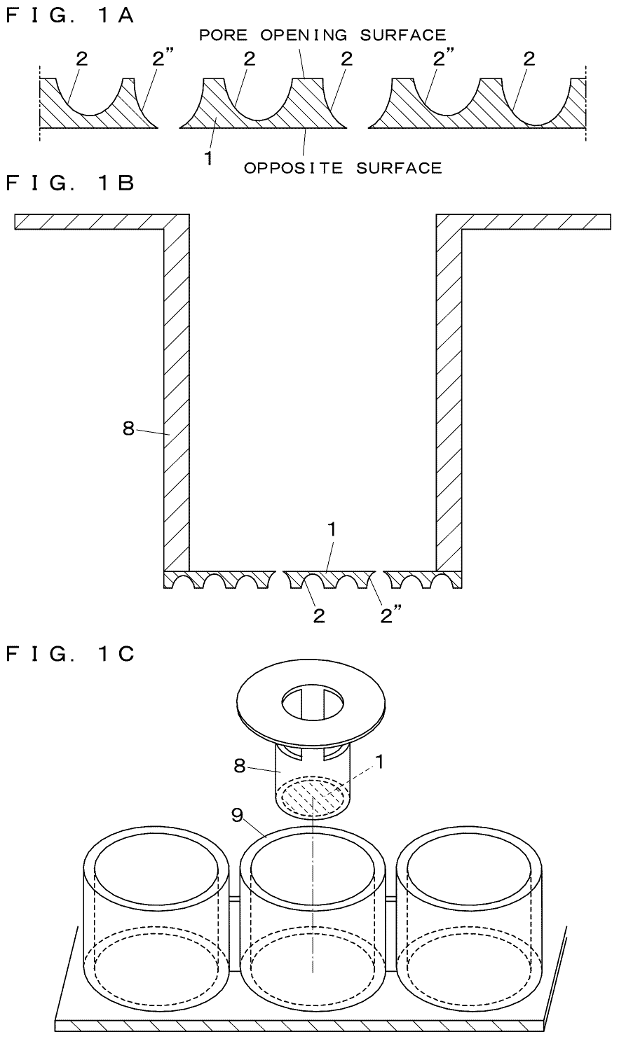

Then, the following PU porous membrane 1 shown in FIG. 1A was selected and used for each sample.
The membrane thickness was 4 μm to 6 μm.
The PU porous membrane 1 had cone-shaped pores 2 that were open on one surface and contracted in diameter inside the membrane. Hereinafter, one surface will be referred to as the “pore opening surface” and the other surface will be referred to as the “opposite surface.”
The number of pores 2 was 5.2E+04 to 2.3E+05/cm².
10% or more of the total number of pores 2 were through-holes 2″ that were contracted in diameter and opened on the opposite surface. This percentage was a value determined by observing a cross section of the PU porous membrane 1 in an arbitrarily selected field of view of an SEM image magnified 1,000 times using a scanning electron microscope (SEM).
The average pore size of the pores 2 observed on the pore opening surface was 5 μm to 8 μm. The average pore size was a value determined by measuring the maximum length of all pores 2 (the maximum value of the lengths of the sides of the quadrangle circumscribing the pore) observed in an arbitrarily selected field of view of an SEM image in which the pore opening surface was magnified 1,000 times using an SEM, and averaging the results.
The average pore size of the through-holes 2″ observed on the opposite surface was 2.52 μm to 3.81 μm. The average pore size was a value determined by measuring the maximum length of all through-holes 2″ (the maximum value of the lengths of the sides of the quadrangle circumscribing the pore) observed in an arbitrarily selected field of view of an SEM image in which the opposite surface was magnified 1,000 times using an SEM, and averaging the results.

Then, as shown in FIG. 1B, the PU porous membrane 1 was fixed to the bottom of an insert 8 with the pore opening surface facing down and the opposite surface facing up. The inner diameter of the insert 8 was 6.6 mm. As shown in FIG. 1C, the insert 8 was inserted into a well 9.
(Sample 1) Sample 1 was obtained by using the PU porous membrane 1 fixed to the insert 8 in this manner as a cell culture membrane without change (with no coating material).

Next, the PU porous membrane fixed to the insert 8 was coated with various coating materials to prepare cell culture membrane samples 2 to 8. First, respective materials used for coating will be described in detail.
As the fibronectin, product name “Human Plasma Fibronectin” (commercially available from Thermo Fisher Scientific) was used. This product was a large glycoprotein and had cell adhesion molecules with a molecular weight of 210 kDa to 250 kDa.
As the collagen, product name “collagen type 1-A” or “collagen type 1-C” (commercially available from Nitta Gelatin, Inc.) was used.
As the extracellular matrix protein, product name “iMatrix-511” (commercially available from Nippi, Inc.) was used. This product was a recombinant protein having the same sequence as an enzymatically degraded fragment of Laminin 511 composed of α5 chain, β1 chain, and γ1 chain.
As the agarose, product name “SeaPlaque agarose” (commercially available from Lonza K. K.) was used. This product was a low-melting-point polysaccharide.

As the phosphate-buffered saline (hereinafter referred to as “PBS”), product number: 10010-023 (pH 7.4 (1X)) (commercially available from Life Technology Ltd) was used.
As the basal medium, Dulbecco's Modified Eagle Medium (hereinafter referred to a “DMEM”) was used. This medium was a basal medium that was widely used for supporting proliferation of various mammalian cells.

### (Sample 2) The Coating Material was a Dried Fibronectin Product

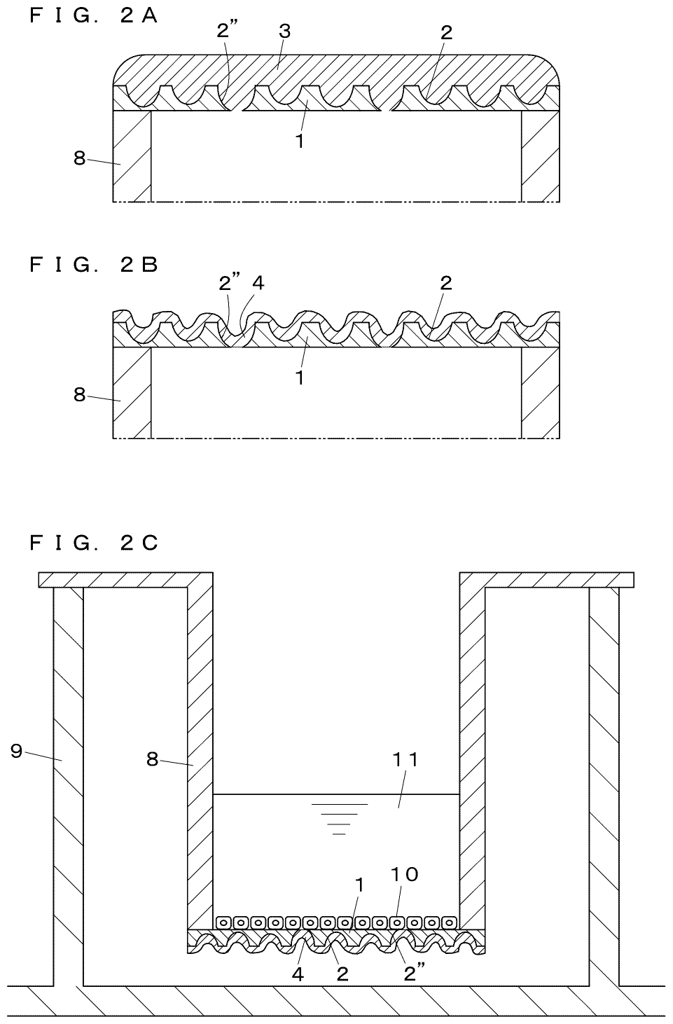

“Human Plasma Fibronectin” was dissolved in PBS to prepare 33 μg/mL of a fibronectin solution.
As shown in FIG. 2A, 140 μL of this fibronectin solution 3 was applied to the pore opening surface of the PU porous membrane 1 of the insert 8 inverted and air-dried for 2 days, and as shown in FIG. 2B, a coating material formed of a dried fibronectin product 4 (in a semi-dry state) was formed on the pore opening surface, and thereby a cell culture membrane of Sample 2 was obtained.

### (Sample 3) The Coating Material was a Collagen Gel

Each solution included in the kit was mixed with “collagen type 1-A” according to the protocol of a collagen gel culture kit (product number: 63800781, commercially available from Nitta Gelatin, Inc.) to prepare a collagen in a gel state.
After the lower side of the PU porous membrane was immersed in this collagen in a gel state for about 1 second, the PU porous membrane was inverted, and left for 20 minutes, a coating material formed of a collagen gel was formed on the pore opening surface (almost the same as in FIG. 2B), and thereby a cell culture membrane of Sample 3 was obtained.

### (Sample 4) The Coating Material was an Agarose Gel (0.5%, Dip Method)

“SeaPlaque agarose” was dissolved in a DMEM to prepare a 0.5% agarose solution.
After the lower side of the PU porous membrane was immersed in this agarose solution for about 1 second, the PU porous membrane was inverted and left until the agarose solidified into a gel (Dip method), a coating material formed of a dried agarose product was formed on the pore opening surface (almost the same as in FIG. 2B), and thereby a cell culture membrane of Sample 4 was obtained.

### (Sample 5) The Coating Material was an Agarose Gel (2%, Dip Method)

“SeaPlaque agarose” was dissolved in a DMEM to prepare a 2% agarose solution.
After the lower side of the PU porous membrane was immersed in this agarose solution for about 1 second, the PU porous membrane was inverted and left until the agarose solidified into a gel (Dip method), a coating material formed of an agarose gel was formed on the pore opening surface (almost the same as in FIG. 2B), and thereby a cell culture membrane of Sample 5 was obtained.

### (Sample 6) The Coating Material was a 2% Agarose Gel (2%, Solidified in the Well)

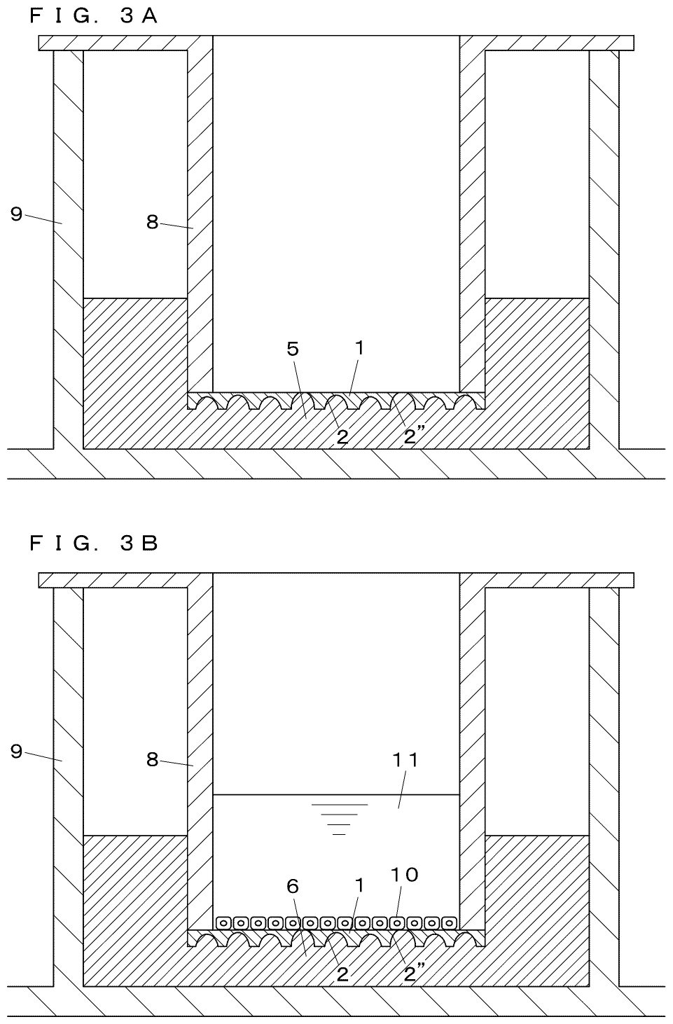

“SeaPlaque agarose” was dissolved in a DMEM to prepare a 2% agarose solution.
As shown in FIG. 3A, 500 μL of this agarose solution 5 was put into the well 9, and the PU porous membrane 1 was set therein, and as shown in FIG. 3B, the agarose solution 5 was solidified into an agarose gel 6 in the well 9 and adhered to the bottom surface of the PU porous membrane 1, and thereby a cell culture membrane of Sample 6 was obtained.

### (Sample 7) The Coating Material was a Collagen Solution Membrane

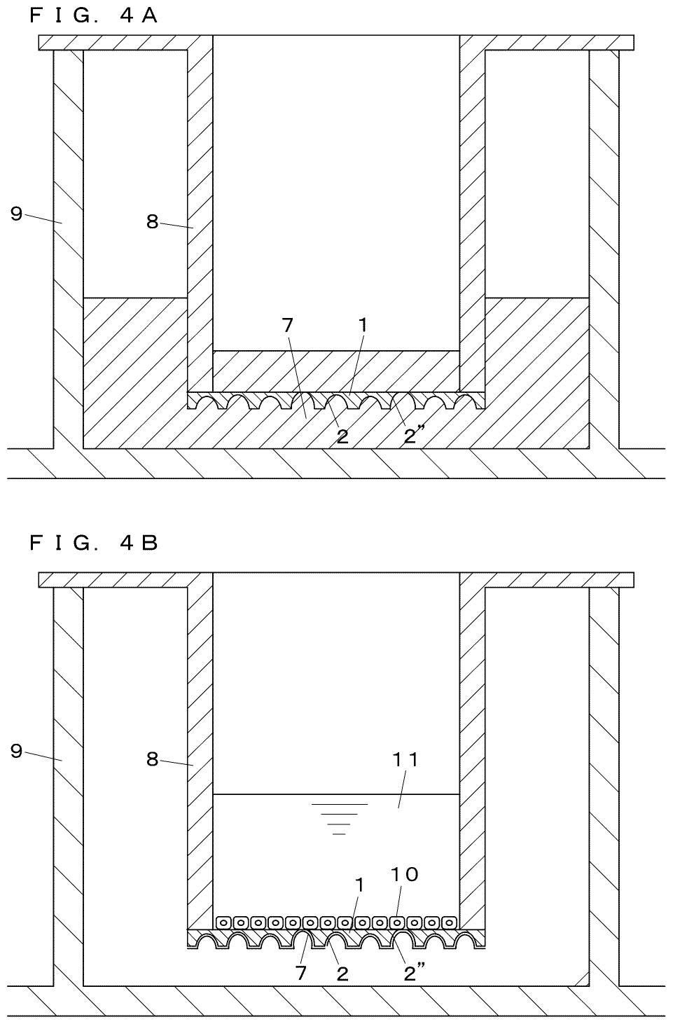

The PU porous membrane was preconditioned overnight (membrane defoaming treatment) with PBS.
“Collagen type 1-C” was diluted with 0.02 N hydrochloric acid to prepare a 200 μg/mL collagen solution.
As shown in FIG. 4A, 100 μL of this collagen solution 7 was added to the top of the PU porous membrane 1 and 450 μL thereof was added to the bottom of the PU porous membrane 1, and left at room temperature for 30 minutes or longer, and as shown in FIG. 4B, immediately before cells were seeded, the excess collagen solution 7 that was not adsorbed was removed, and thereby a cell culture membrane of Sample 7 was obtained.

### (Sample 8) The Coating Material was a Matrix Protein Solution Membrane

The PU porous membrane was preconditioned overnight with PBS.
“iMatrix-511” was dissolved in PBS to prepare a 5 μg/mL matrix protein solution.
200 μL of this matrix protein solution was added to the top of the PU porous membrane and 500 μL thereof was added to the bottom of the PU porous membrane, and left at room temperature for 3 hours or longer (almost the same as in FIG. 4A), and immediately before cells were seeded, the excess matrix protein solution that was not adsorbed was removed, and thereby a cell culture membrane of Sample 8 (almost the same as in FIG. 4B) was obtained.

Here, two or more samples were prepared for each of Samples 1 to 8.

### <2> Preparation of Sample Using PC Porous Membrane

As the polycarbonate (PC) porous membrane, one having a pore diameter of 3.0 μm (product name “Transwell,” commercially available from Corning Inc.) was used.
This PC porous membrane that was used as a cell culture membrane without change was set as Sample 1 (not shown). In addition, this PC porous membrane was coated with the same coating material as in the above <1> PU porous membrane samples 2 to 8 using the same method to prepare cell culture membrane samples 2 to 8 (not shown).

### <3> Monolayer Culture of Cells

Caco-2 cells 10 (5×10⁴ cells) were seeded on the top surface (the opposite surface) of the above <1><2> cell culture membrane samples 1 to 8, DMEM was added as a medium 11, and the cells were cultured for 7 days (FIG. 2C, FIG. 3B, and FIG. 4B). The medium 11 was replaced after 3 days and after 5 days. Caco-2 cells 10 were cultured so that they spread on the top surface of the cell culture membrane in all samples, but depending on the sample, they sometimes went around to the bottom surface of the cell culture membrane.

### <4> Measurement and Evaluation

### (a) Observation of Going Around of Cells

After culture was completed, in one sample, cell nuclei were stained using 4′, 6-diamidino-2-phenylindole (hereinafter referred to as “DAPI”), and in another sample, cytoskeletons were stained using phalloidin. After staining, when cells on the top surface of the cell culture membrane were scraped off with a cell scraper, and observation was performed from the bottom side of the cell culture membrane, it was determined whether cell nuclei and pseudopods went around due to their passing through the through-holes of the cell culture membrane from the upper side to the lower side.
Here, in staining using DAPI, a blue fluorescent dye was bound to DNA in cultured cells and emitted blue fluorescence, and thus cell nuclei could be identified. In staining using phalloidin, cytoskeletons (actin filaments, etc.) of cultured cells and tissue sections were stained by labeling them with a green fluorescent dye.

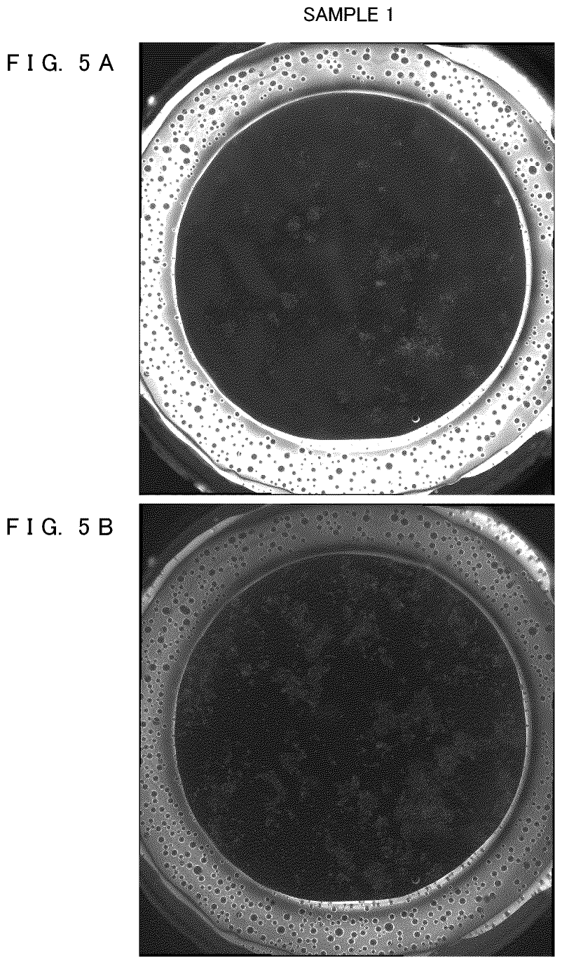

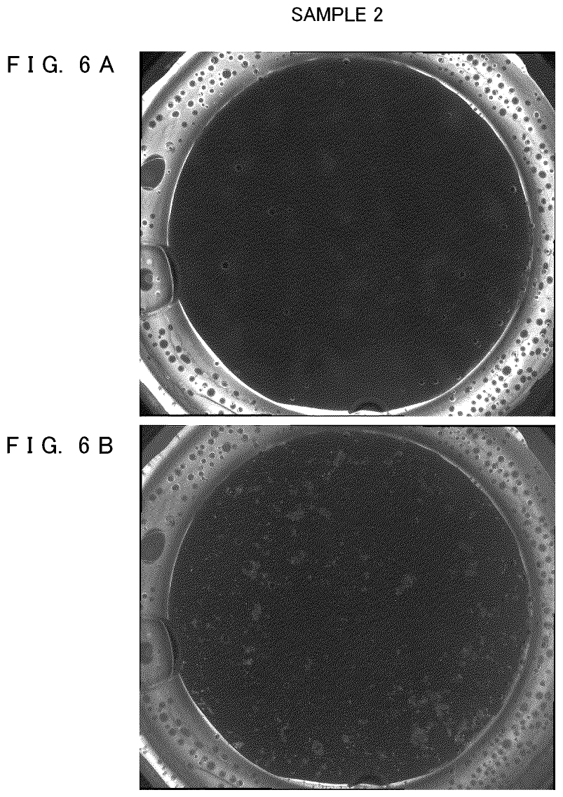

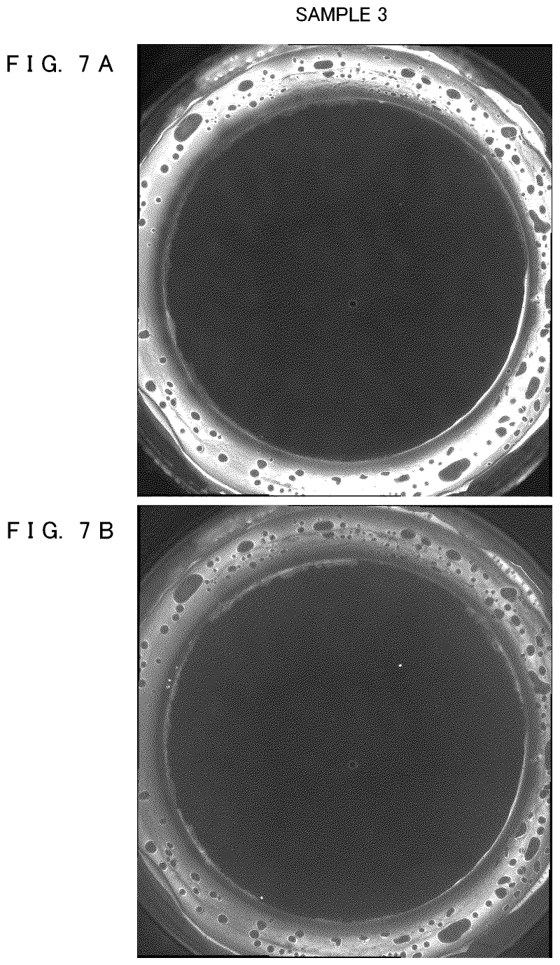

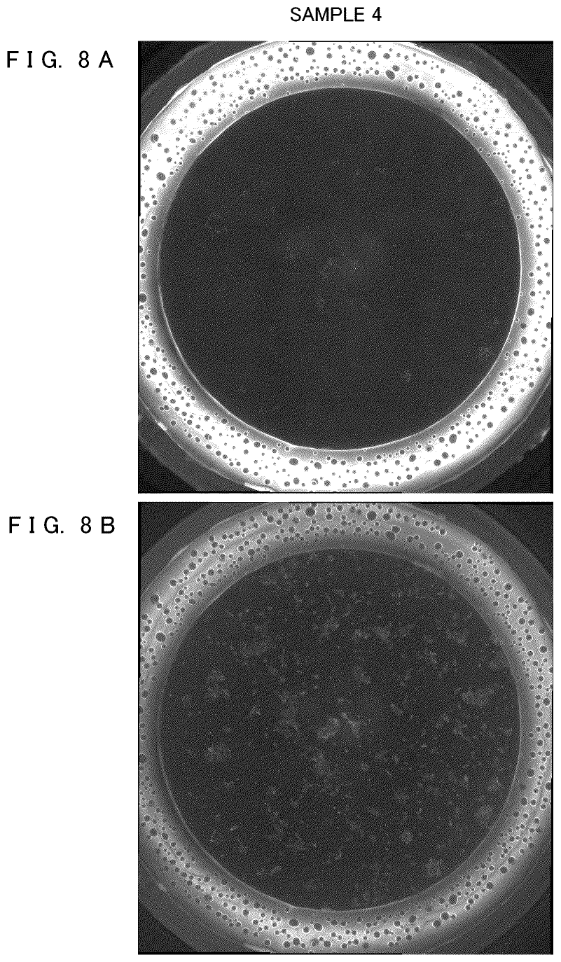

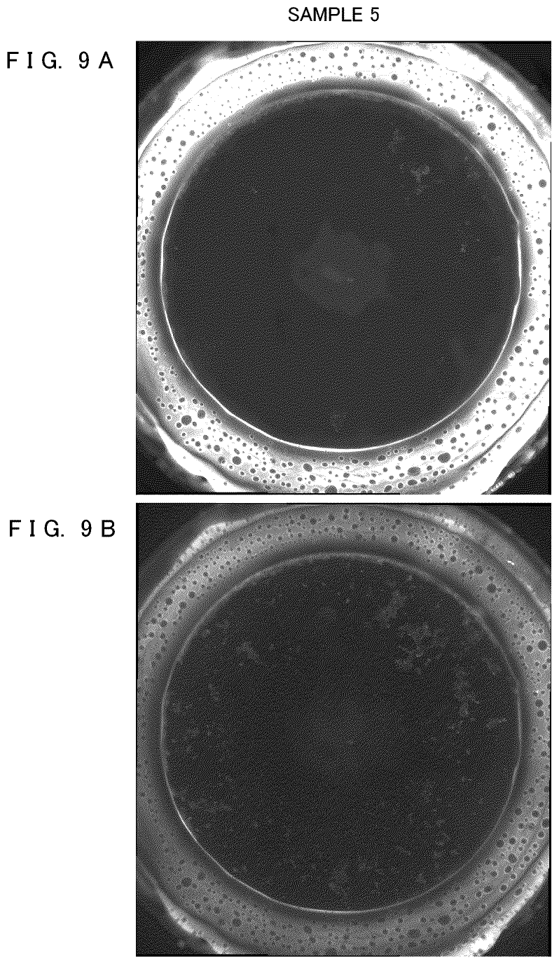

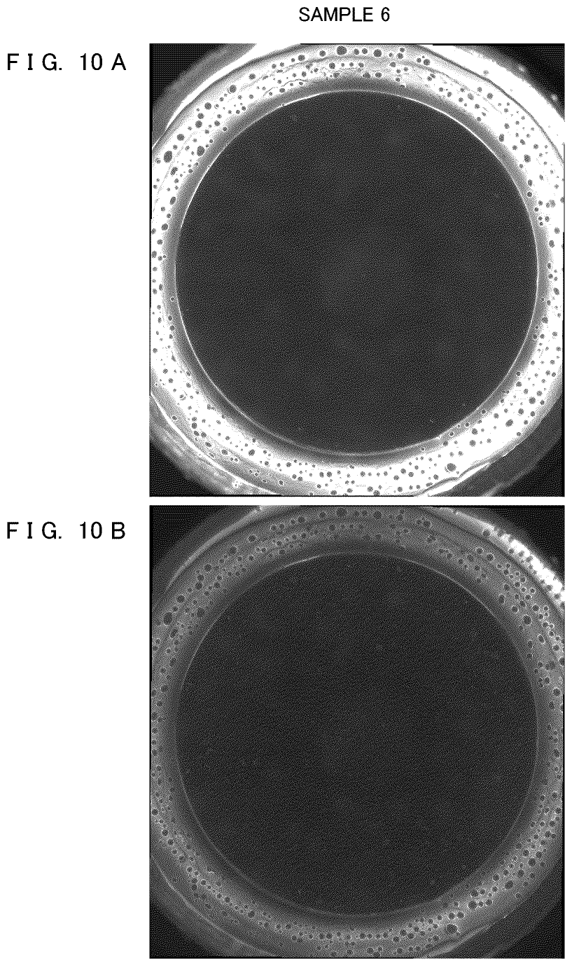

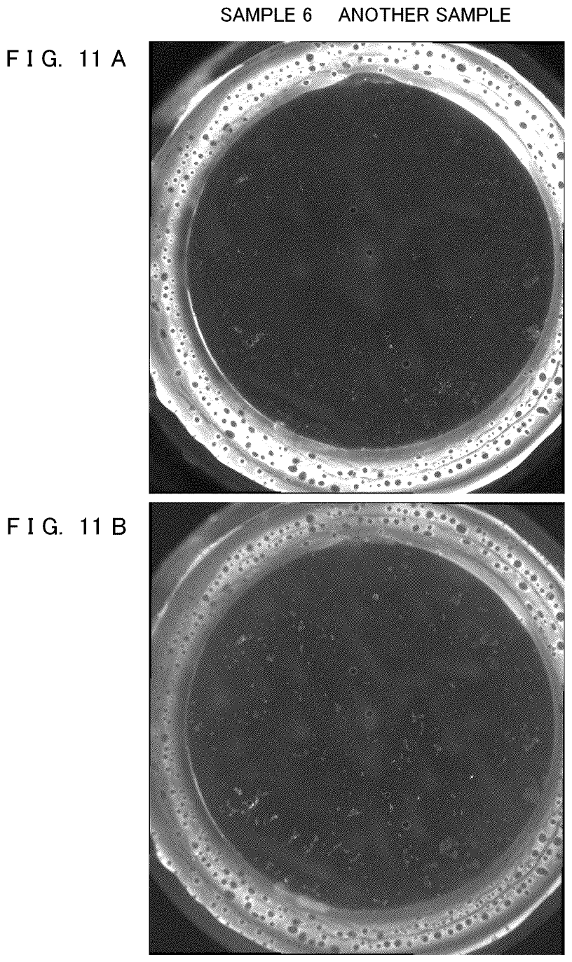

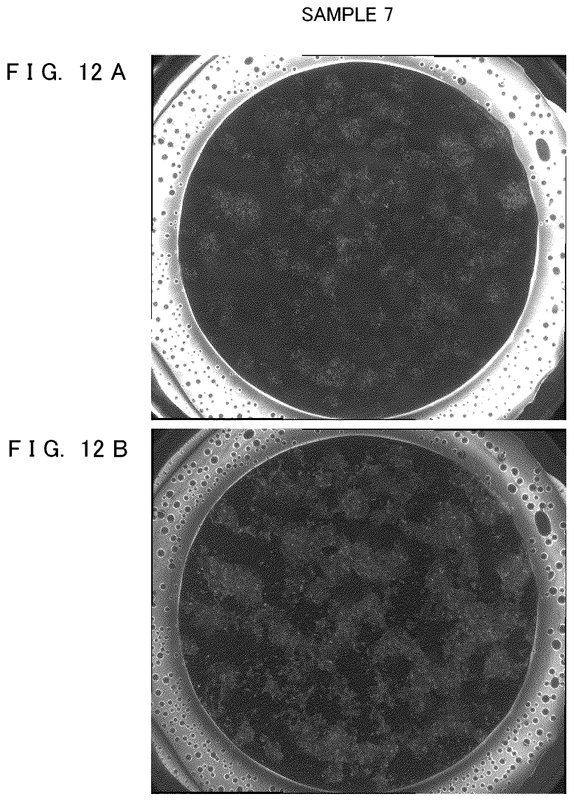

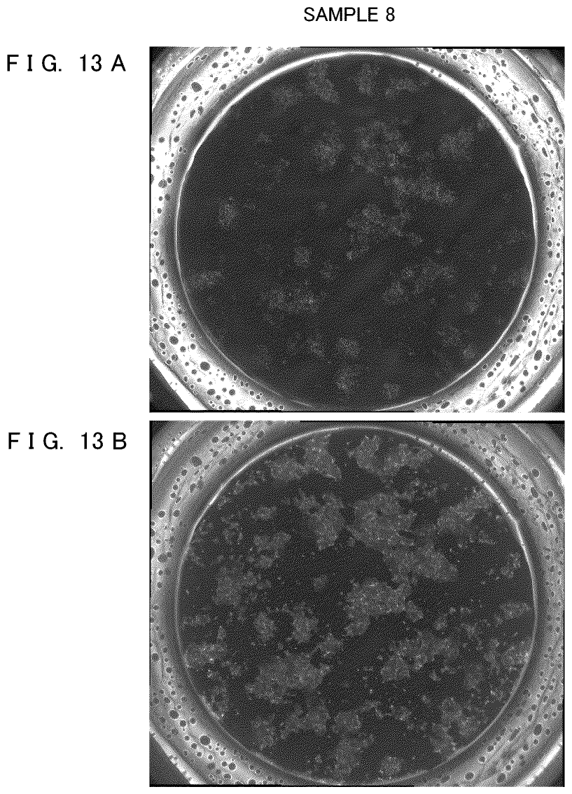

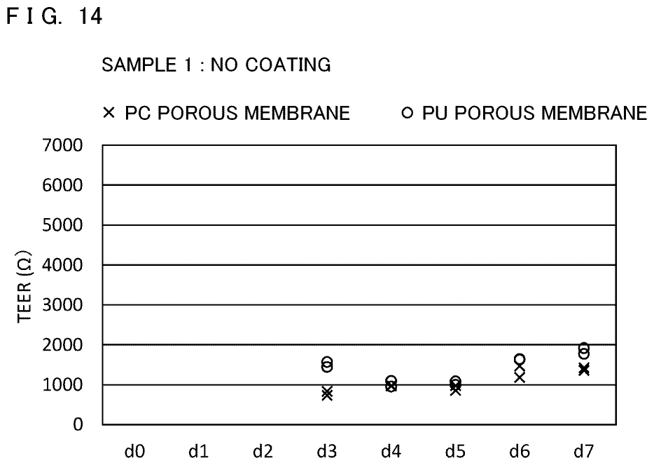

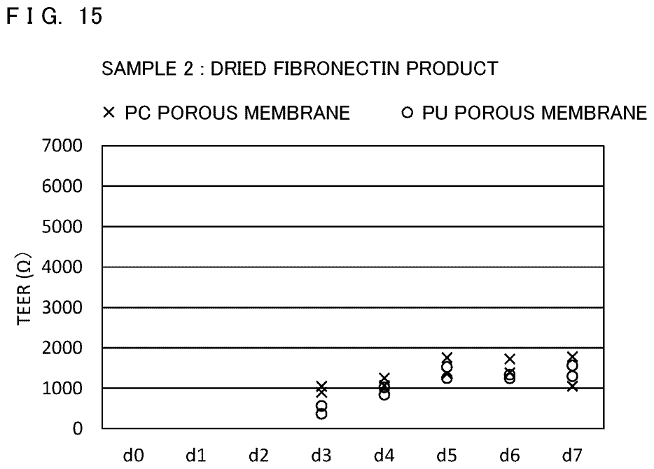

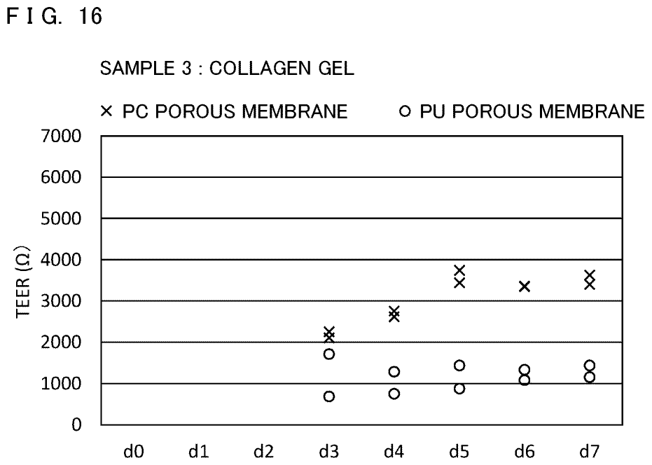

As shown in the above [BRIEF DESCRIPTION OF DRAWINGS], FIGS. 5A to 13B show stained images of Samples 1 to 8 using the PU porous membrane. FIGS. 10A to 11B show stained images of two samples in Sample 6.
As shown in FIGS. 5A and 5B, in Sample 1 (with no coating material), nuclei and pseudopods went around.
As shown in FIGS. 6A and 6B, in Sample 2 (dried fibronectin product), nuclei did not go around, but pseudopods slightly went around.
As shown in FIGS. 7A and 7B, in Sample 3 (collagen gel), nuclei did not go around, and pseudopods did not go around.
As shown in FIGS. 8A and 8B, in Sample 4 (agarose gel (0.5%, Dip method)), nuclei and pseudopods slightly went around.
As shown in FIGS. 9A and 9B, in Sample 5 (agarose gel (2%, Dip method)), nuclei and pseudopods slightly went around. Cells were peeled off of the gel during staining, and there was a possibility that the gel was already peeled off during the culture period.
As shown in FIGS. 10A and 10B, in one of two samples for Sample 6 (agarose gel (2%, solidified in the well)), nuclei and pseudopods did not go around, and as shown in FIGS. 11A and 11B, in the other sample, nuclei and pseudopods slightly went around, and variations were observed in the results.
As shown in FIGS. 12A and 12B, in Sample 7 (collagen solution membrane), nuclei and pseudopods went around, the amount of going around was larger than in Sample 1 (with no coating material).
As shown in FIGS. 13A and 13B, in Sample 8 (matrix protein solution membrane), nuclei and pseudopods went around, and the amount of going around was larger than in Sample 1 (with no coating material).
The above cell going around determination results are summarized in Table 1.

TABLE 1

| | | Presence of | Presence of |
|---|---|---|---|
| | | nuclei | pseudopod |
| | | going | going |
| | Coating material | around | around |
| Sample | None | ++ | ++ |
| 1 | | | |
| Sample | Dried fibronectin product | − | + |
| 2 | | | |
| Sample | Collagen gel | − | − |
| 3 | | | |
| Sample | Agarose gel (0.5%, Dip method) | + | + |
| 4 | | | |
| Sample | Agarose gel (2%, Dip method) | + | + |
| 5 | | | |
| Sample | Agarose gel (2%, solidified | −/+ | −/+ |
| 6 | in the well) | | |
| Sample | Collagen solution membrane | +++ | +++ |
| 7 | | | |
| Sample | Matrix protein solution | +++ | +++ |
| 8 | membrane | | |

−: no going around
+: very small amount of going around
++: small amount of going around
+++: large amount of going around

For Samples 1 to 8 using the PC porous membrane, going around of cell nuclei and pseudopods observed from the stained images (not shown) was generally the same as the results of Samples 1 to 8 using the PU porous membrane.

Based on the above results, using each Sample 1 with no coating material in which the PU porous membrane or the PC porous membrane was used without change as a comparative example, the coating material of Samples 2 to 6 could be said to be a solid coating material that suppresses cells from passing through the through-holes, and therefore the cell culture membranes of Samples 2 to 6 were regarded as examples of the present invention.

On the other hand, the coating materials of Samples 7 and 8 could not be said to be a solid coating material that suppresses cells from passing through the through-holes, and therefore the cell culture membranes of Samples 7 and 8 were regarded as reference examples.

### (b) TEER Value

From the third day to the seventh day after culture started, the trans-epithelial electrical resistance (hereinafter referred to as “TEER value”) was measured and its variation was examined. However, in Sample 6, the lower layer of the membrane was a solid medium, no TEER electrode was inserted, and thus the TEER value was measured only on the seventh day. The TEER value is an index for measuring the barrier function of a cell monolayer non-invasively and overtime, and is an important evaluation item in drug discovery screening.

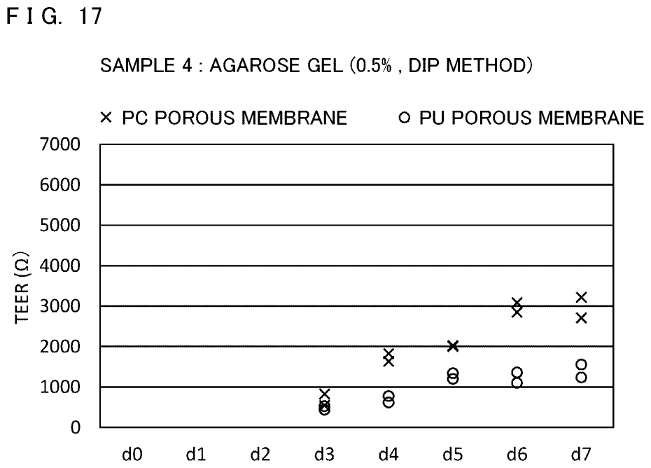

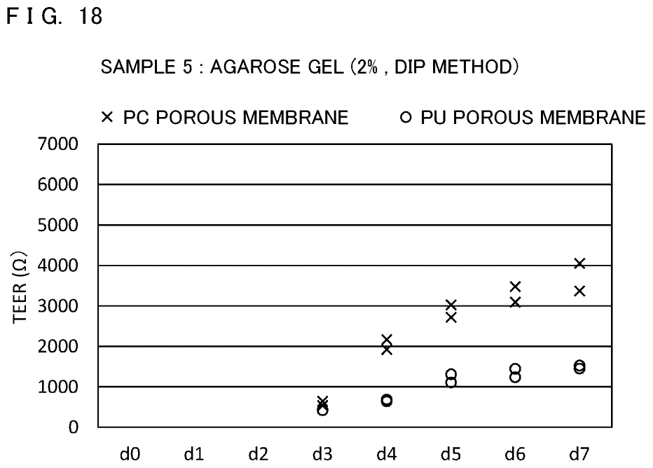

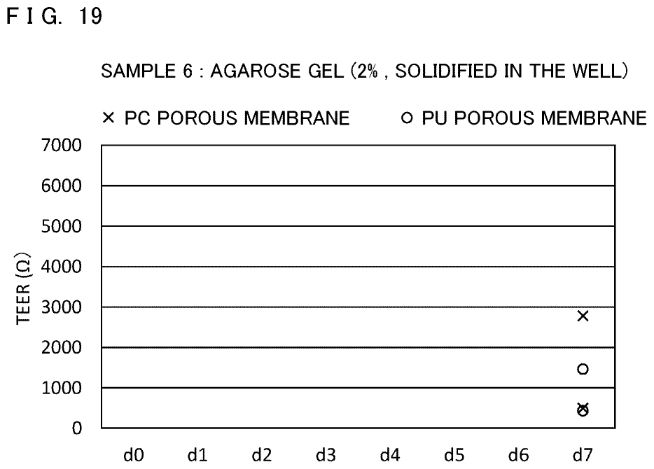

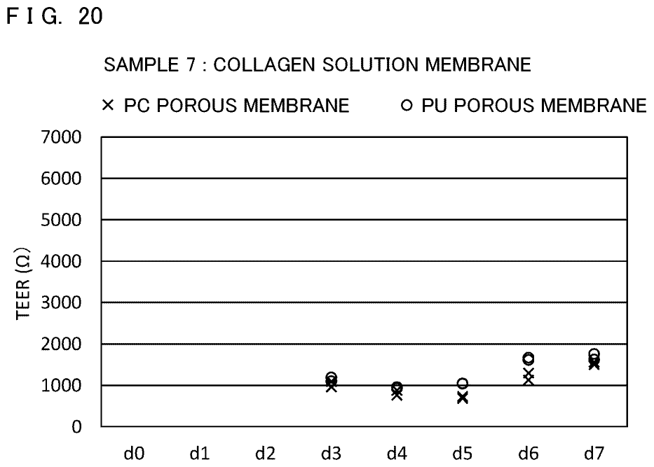

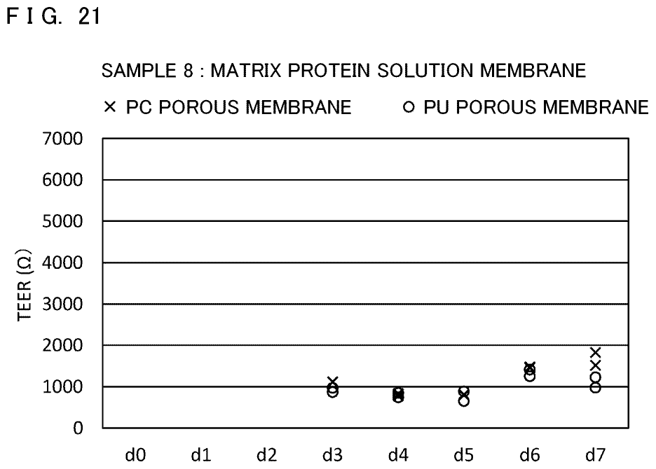

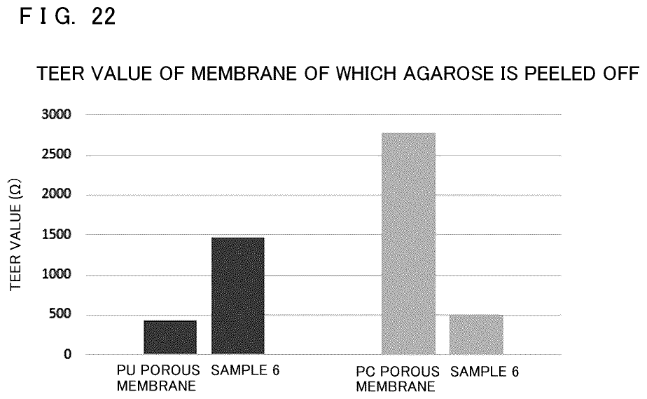

As shown in the above [BRIEF DESCRIPTION OF DRAWINGS], FIG. 14 to FIG. 21 show the variation in the TEER value of Samples 1 to 8 using the PU porous membrane or the PC porous membrane.
In any of the methods, the value was lower than or almost the same as that of commercially available standard membranes. However, Sample 6 was measured after being peeled off of the agarose during measurement. In some samples, a strong force was required to peel off the membrane and the agarose, and in these samples, as shown in FIG. 22, the TEER value was low. Since the value was as low as 500 Ω or less, it was thought that cells were damaged and leaked when the agarose was peeled off.

### <5> Consideration

(a) It was thought that one of the reasons why the solid coating materials of Samples 2 to 6 suppressed cells from passing through the through-holes was the occurrence of cell contact inhibition. That is, there is a known property of cells stopping proliferation when the space in which the cells can proliferate is filled when monolayer cells are proliferated. This is thought to be because cells which allow the through-holes to come into contact with a solid such as a dried fibronectin product or a collagen gel stopped proliferation and were prevented from moving to the opposite surface.

(b) It was found that, compared to solid coating materials of Samples 2 to 6, the coating materials of Samples 7 and 8, which remained as a liquid and were not cured, did not have an effect of suppressing cells from passing through the through-holes. The reason why the amount of going around was larger in Samples 7 and 8 than Sample 1 was thought to be because the coating materials, which remained as a liquid and were not cured, had strong adhesion to receptors (integrin) on the cell membrane and improved proliferation properties.

(c) Although agarose gels of Samples 4 to 6 were solids, the reason why a small amount of nuclei went around was speculated as follows. That is, it was thought that, since the agarose gel had a large amount of syneresis, syneresis from the gel entered between the membrane and the gel, the adhesiveness was weakened, and a space in which cells could move was created. Therefore, a coating material with a small amount of syneresis such as a collagen gel is considered more preferable.

### <6> Two-Layer Culture and Monolayer Culture of Cells

When PU porous membranes or PC porous membranes regarded as the first and second examples were used, and
a step of using the first PU porous membrane or PC porous membrane as a cell culture membrane (corresponding to each Sample 1), and performing two-layer culture of different types of cells on both surfaces of the cell culture membrane, and
a step of coating any one surface of the second PU porous membrane or PC porous membrane with a solid coating material that suppresses cells from passing through the through-holes to form a cell culture membrane (corresponding to Samples 2 to 6), and performing monolayer culture of cells on one surface of the cell culture membrane were performed,
it was possible to perform monolayer culture as opposed to two-layer culture based on the same PU porous membrane or PC porous membrane.

Here, the present invention is not limited to the examples, and can be embodied by being appropriately changed without departing from the spirit and scope of the invention.

### REFERENCE SIGNS LIST

1 PU porous membrane
2 Pore
2″ Through-hole
3 Fibronectin solution
4 Dried fibronectin product
5 Agarose solution
6 Agarose gel
7 Collagen solution
8 Insert
9 Well
10 Cell
11 Medium

1. A cell culture membrane, comprising:
a resin porous membrane formed of a resin and having a plurality of pores that is open on at least one surface, in which at least some of the pores are through-holes that penetrate in a membrane thickness direction; and
a solid coating material that is applied to any one surface of the resin porous membrane and suppresses cells from passing through the through-holes.

2. The cell culture membrane according to claim 1,
wherein the resin is a polyurethane, and the pores and the through-holes have a cone shape.

3. The cell culture membrane according to claim 1,
wherein the coating material is a dried glycoprotein product.

4. The cell culture membrane according to claim 2,
wherein the coating material is a dried glycoprotein product.

5. The cell culture membrane according to claim 1,
wherein the coating material is a protein gel.

6. The cell culture membrane according to claim 2,
wherein the coating material is a protein gel.

7. The cell culture membrane according to claim 1,
wherein the coating material is a polysaccharide gel.

8. The cell culture membrane according to claim 2,
wherein the coating material is a polysaccharide gel.

9. A cell culture method using a resin porous membrane formed of a resin and having a plurality of pores that is open on at least one surface, in which at least some of the pores are through-holes that penetrate in a membrane thickness direction, the cell culture method comprising
coating any one surface of the resin porous membrane with a solid coating material that suppresses cells from passing through the through-holes to form a cell culture membrane, and performing monolayer culture of cells on one surface of the cell culture membrane.

10. A cell culture method using first and second resin porous membranes formed of a resin and having a plurality of pores that is open on at least one surface, in which at least some of the pores are through-holes that penetrate in a membrane thickness direction, the cell culture method comprising:
a step of using the first resin porous membrane as a cell culture membrane and performing two-layer culture of different types of cells on both surfaces of the cell culture membrane; and
a step of coating any one surface of the second resin porous membrane with a solid coating material that suppresses cells from passing through the through-holes to form a cell culture membrane, and performing monolayer culture of cells on one surface of the cell culture membrane.
